(19) United States
(12) Patent Application Publication
(10) Pub. No.: US 2012/0238003 A1
(43) Pub. Date: Sep. 20, 2012

(54) MICROORGANISM CONCENTRATING APPARATUS AND METHOD

(76) Inventors: Taro Fukaya, Tokyo (JP); Kenji Tsutsumi, Yokohama-shi (JP); Atsushi Yamazaki, Tokyo (JP); Ichiro Yamanashi, Tokyo (JP); Shuji Seki, Yokohama-shi (JP)

(21) Appl. No.: 13/197,361

(22) Filed: Aug. 3, 2011

(30) Foreign Application Priority Data
Mar. 14, 2011 (JP) 2011-055803

(51) Int. Cl. C12M 1/12; C12N 1/02

(52) U.S. Cl. 435/261; 435/308.1

(57) ABSTRACT

A microorganism-concentrating apparatus according to one embodiment includes a mixing device, a filtering device, and a magnetic separation device. The mixing device is configured to mix an aqueous suspension of microorganisms with a particulate filter aid includes magnetic particles to prepare an aqueous slurry of the microorganisms and the particulate filter aid. The filtering device is configured to filter the aqueous slurry supplied from the mixing device to provide a filter cake includes the microorganisms and the particulate filter aid, and to provide a filtrate. The magnetic separation device is configured to magnetically separate the filter cake supplied from the filtering device into the microorganisms and the particulate filter aid.

### CROSS-REFERENCE TO RELATED APPLICATIONS

This application is based upon and claims the benefit of priority from prior Japanese Patent Application No. 2011-055803, filed Mar. 14, 2011, the entire contents of which are incorporated herein by reference.

### FIELD

Embodiments described herein relate generally to a microorganism concentrating apparatus and method.

### BACKGROUND

Among water microorganisms represented by algae, there are many microorganisms which produce useful substances, and these microorganisms have been cultured to produce such useful substances. Examples of such useful substance-producing microorganisms include chlorella utilized in health foods and oil-producing bacteria which may be used as non-fossil fuels.

In order to effectively utilize these microorganisms, concentration is required after the cultivation. Also, microorganisms, other than these useful substance-producing microorganisms, such as E. coli, viruses, present in a dilute concentration, are required to be concentrated for the purpose of biological investigation. Concentration can be effected by, e.g., centrifugal separation or membrane separation. However, recently, magnetic separation which uses magnetic particles attracts attention as a method of concentrating microorganisms which is energy-saving and achieves a high concentration/separation efficiencies. However, prior art magnetic separation method is not satisfactory in terms of separation/concentration speed.

### BRIEF DESCRIPTION OF THE DRAWINGS

FIG. 1 is a schematic view illustrating a microorganism-concentrating apparatus according to one embodiment; and

FIG. 2 is a schematic view illustrating a magnetic separation device which may be used in the concentrating apparatus of FIG. 1.

### DETAILED DESCRIPTION

In general, a microorganism-concentrating apparatus according to one embodiment comprises a mixing device, a filtering device, and a magnetic separation device. The mixing device is configured to mix an aqueous suspension of microorganisms with a particulate filter aid comprising magnetic particles to prepare an aqueous slurry of the microorganisms and the particulate filter aid. The filtering device is configured to filter the aqueous slurry supplied from the mixing device to provide a filter cake comprising the microorganisms and the particulate filter aid, and to provide a filtrate. The magnetic separation device is configured to magnetically separate the filter cake supplied from the filtering device into the microorganisms and the particulate filter aid.

Further, a microorganism concentrating method according to one embodiment comprises mixing an aqueous suspension of microorganisms with a particulate filter aid comprising magnetic particles to prepare an aqueous slurry of the microorganisms and the particulate filter aid. The aqueous slurry is filtered through a filter medium to provide a filter cake comprising the microorganisms and the particulate filter aid, and to provide a filtrate, wherein the filter cake is provided on the filter medium. Then, the filter cake is magnetically separated into the microorganisms and the particulate filter aid.

Various embodiments of a microorganism-concentrating apparatus and method will be described below with reference to the drawings.

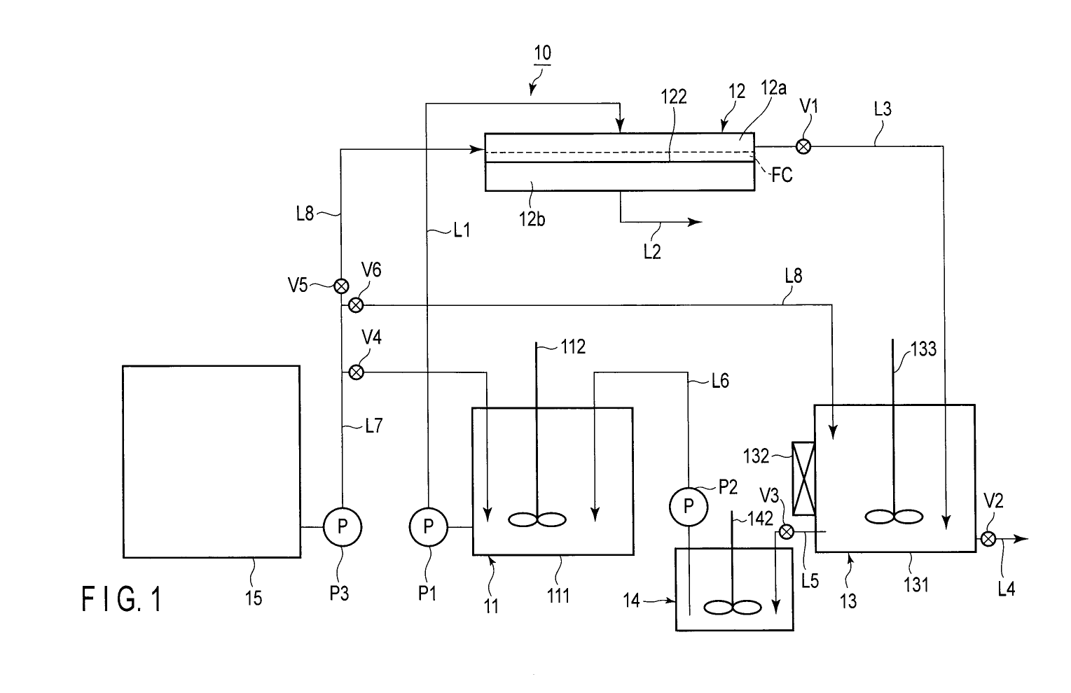

FIG. 1 schematically illustrates a microorganism-concentrating apparatus 10 according to one embodiment. The concentrating apparatus is connected to a microorganism-culturing tank 15.

The concentrating apparatus comprises a mixing device 11, a filtering device 12, and a magnetic separation device 13.

The mixing device 11 comprises a mixing tank 111 which receives water containing microorganisms, i.e., an aqueous suspension of microorganisms, and a particulate filter aid comprising magnetic particles and in which they are mixed together. Within the mixing tank 111, a stirrer 112 is arranged, which stirs and mixes the aqueous suspension of microorganisms, and the particulate filter aid to prepare an aqueous slurry comprising the microorganisms and the particulate filter.

The filtering device 12 comprises an envelope (rectangular parallelepiped shape in FIG. 1). The inner space of the envelope 121 is partitioned by a filter medium 122 into two spaces, i.e., an upper space 12a and a lower space 12b. In FIG. 1, the filter medium is placed horizontally.

The mixing tank 111 of the mixing device 11 and the upper space 12a of the filtering device 12 are communicated with each other through a line L1 provided with a pump P1. The aqueous slurry containing the microorganisms and the particulate filter aid prepared by the mixing device 11 is introduced into the upper space 12a of the filtering device 12 through the line L1 by driving the pump P1, is filtered through the filter medium 122, and forms, on the filter medium 122, a filter cake FC containing the microorganisms and the particulate filter aid. The filtrate is discharged through a line L2 which communicates with the lower space 12b of the filtering device 12.

The magnetic separation device 13 comprises a separation tank which receives the filter cake containing the microorganisms and the particulate filter aid from the filtering device 12 and in which the filter cake is magnetically separated. On the outer peripheral wall of the separation tank 131, an electromagnet 132 is arranged, which is connected to a power source not illustrated. In the separation tank 131, a stirrer 133 is arranged, which stirs the filter cake. The filter cake containing the microorganisms and the particulate filter aid supplied to the separation tank 131 is separated into the particulate filter aid and the microorganisms (and water) by the particulate filter aid being magnetically held on that inner wall portion of the separation tank 131 which corresponds to the electromagnet 132 by driving the electromagnet 132. The microorganisms (slurry) separated and concentrated by the magnetic separation device 13 are removed through a line L4 provided with an on-off valve V2.

The upper space 12a of the filtering device 12 and the separation tank 131 of the magnetic separation device 13 are communicated with each other through a line L3 provided with an on-off valve V1. The filter cake discharged from the upper space 12a of the filtering device 12 is introduced into the magnetic separation device 13 through the line L3 by opening the on-off valve V1.

A filter aid accommodating tank 14 is arranged upstream of the mixing device 11 and downstream of the magnetic separation device 13. A stirrer is arranged within the tank 14. The filter aid accommodating tank 14 and the mixing device are communicated with each other through a line L6 provided with a pump P2. The particulate filter aid within the filter aid accommodating tank 14 is supplied into the mixing tank 111 of the mixing device 11 through the line L6 by driving the pump P2. Also, the separation tank 131 and the filter aid accommodating tank 14 are communicated with each other through a line L5 provided with an on-off valve V3. The particulate filter aid separated from the microorganisms in the separation tank 131 is supplied to the filter aid accommodating tank 14 through the line L5, and is reused.

As noted above, the concentrating apparatus 10 is connected to the culturing tank 15 in which microorganisms are cultured. More specifically, the culturing tank 15 and the mixing tank 111 of the mixing device 11 are communicated with each other through a line L7 provided with a pump P3 and an on-off valve V4. By opening the on-off valve V4 and driving the pump P3, the aqueous suspension of microorganisms in the culturing tank 15 is supplied into the mixing tank 111 of the mixing device 11.

Further, a line L8 is provided, which is branched from the line L7 downstream of the on-off valve V4, and communicates with the upper space 12a of the filtering device 12. The line L8 is provided with an on-off valve V5. By opening the on-off valve V5 and driving the pump P3, the aqueous suspension of microorganisms in the culturing tank 15 flows through the lines L7 and L8, and wash out the filter cake FC deposited on the filter medium 121 of the filtering device 12.

Furthermore, a line L9 is provided, which is branched from the line L8 upstream of the on-off valve V5, and communicates with the separation tank 131 of the magnetic separation device 13. The line L9 is provided with an on-off valve V6. By opening the on-off valve V6 and driving the pump P3, the aqueous suspension of microorganisms in the culturing tank 15 is supplied into the separation tank 131 through the line L9, and washes the particulate filter aid separated from the microorganisms in the separation tank 131 to prepare an aqueous slurry of the particulate filter aid.

A method of concentrating microorganisms using the microorganism-concentrating apparatus illustrated in FIG. 1 will be described below

Before operation, all of the on-off valves V1-V6 are closed, and all of the pumps P1-P3 are not driven. The culturing tank 15 contains microorganisms cultivated in water, i.e., an aqueous suspension of microorganisms. In addition, the filter aid accommodating tank 14 contains an aqueous slurry of the particulate filter aid which is being stirred by the stirrer 142.

In the concentration operation of the microorganisms, the on-off valve V4 is opened and the pump P3 is driven to supply the aqueous suspension of microorganisms into the mixing tank 111 of the mixing device 11 through the line L7, while the aqueous slurry of the particulate filter aid is supplied from the filter aid accommodating tank 14 into the mixing tank 111 through the line L6. In the mixing tank 111, the supplied aqueous suspension of microorganisms and aqueous slurry of the particulate filter aid are stirred and mixed by the stirrer 112. When a predetermined amount of aqueous slurry of the microorganisms and the particulate filter aid has been prepared in the mixing tank 111, the pumps P2 and P3 are deactivated and the on-off valve V4 is closed. Then, by driving the pump P1, the aqueous slurry of the microorganisms and the particulate filter aid prepared in the mixing tank 111 is introduced into the upper space 12a of the filtering device through the line L1. The aqueous slurry of the microorganisms and the particulate filter aid is filtered through the filter medium 122, whereby a filter cake FC containing the microorganisms and the particulate filter aid is formed on the filter medium 122, while the filtrate is discharged from the line L2 through the lower space 12b of the filtering device 12. When a predetermined amount of filter cake is deposited on the filter medium 122, the pump P1 is deactivated. Then, the on-off valve V1 is opened and the on-off valve V5 is also opened, and the pump P3 is driven to supply the aqueous suspension of microorganisms in the culturing tank 15 toward the filter cake FC from the side of the upper space 12a of the filtering device 12, through the lines L7 and L8. The filter cake FC on the filter medium 122 is washed out from the filter medium 122 by the aqueous suspension of microorganisms supplied through the lines L7 and L8 to prepare an aqueous slurry together with the aqueous suspension of microorganisms, which slurry is introduced into the separation tank 131 of the magnetic separation device 13 through the line L3. The amount of aqueous suspension of microorganisms, as aqueous wash-out liquid, supplied from the culturing tank 15 is sufficient to make the concentration of the microorganisms in the aqueous slurry of the filter cake higher than the concentration of the microorganisms in the aqueous suspension of microorganisms in the culturing tank 15. When all the filter cake on the filter medium 122 is removed in this way, the pump P3 is deactivated, and the on-off valves V5 and V1 are closed. In the separation tank 131, the supplied, concentrated aqueous slurry (containing the microorganisms and the particulate filter aid) is sufficiently stirred by the stirrer 133, and then the electromagnet 132 is activated. So, the particulate filter aid in the tank 131 are attracted by the electromagnet 132 and held at that inner wall portion of the tank 132 which corresponds to the electromagnet 132. In this way, the particulate filter aid and the microorganisms are magnetically separated from each other in the separation tank 132. The microorganisms (and water) from which the particulate filter aid has been separated are recovered as a concentrated microorganism slurry from the line L4 by opening the on-off valve V2. When all of the microorganisms are recovered from the separation tank 131, the on-off valve V2 is closed and the electromagnet 132 is deactivated. Then, the particulate filter aid accumulates on the bottom of the separation tank 131. Then, by opening the on-off valve V6 and driving the pump P3, the aqueous suspension of microorganisms is introduced from the culturing tank 15 into the separation tank 131 through the line L9, and is stirred with the particulate filter aid by the stirrer 133. An aqueous slurry of the particulate filter aid thus obtained is supplied into the filter aid accommodating tank 14 through the line L5 by opening the on-off valve V3. In this way the particulate filter aid is re-used.

Next some of the elements of the concentrating apparatus 10 will be explained additionally or in detail, or alternative of the elements will be explained.

<Culturing Tank and Microorganisms>

The concentrating apparatus 10 described with reference to FIG. 1 obtains the aqueous suspension of microorganisms in the culturing tank 15. However, an aqueous suspension of microorganisms may be taken in directly from a pond, a lake, a river, etc. depending on microorganisms to be concentrated or the purpose of concentration. In such a concentrating apparatus, the line L6 is communicated directly with the pond, lake of the river, instead of the culturing tank 15.

In one or more embodiments, the microorganisms to be concentrated are not particularly limited. For example, bacteria such as Escherichia coli, algae such as waterbloom and chlorella, yeasts such as beer yeast, viruses, and oil-producing bacteria may be used. As described below, in one or more embodiments, it is preferred that the particle size of the microorganisms be 0.5 to 5 times the particle size of the particulate filter aid. Further, in view of disperseability in water and linear velocity, the particle size of the particulate filter aide is preferably 1 to 5 μm. Thus, it is suitable that the microorganisms to be concentrate have a particle size of 0.5 to 25 μm.

<Particulate Filter Aid>

In one or more embodiments, the magnetic particles constituting the particulate filter aid is formed of magnetic material. Examples of the magnetic material include iron and an iron alloy, as well as ferritic magnetic materials such as magnetite, ilmenite, pyrrhotite, magnesia ferrite, cobalt ferrite, nickel ferrite and barium ferrite. The ferritic magnetic materials are stable in water, and thus can be suitable used. In particular, magnetite (Fe₃O₄) is inexpensive and stable as magnetic material in water, and its constitutional elements are safe, and thus can be easily used in water treatment. The shape of the magnetic particles is not particularly limited and may be spherical, polyhedral or short fibrous, for example.

In one or more embodiments, the particulate filter aid may consist of magnetic particles (primary particles). Alternatively, the particulate filter aid may consist of aggregates of magnetic particles whose surfaces are covered with a polymer. In the magnetic particle whose surface is covered with a polymer, the magnetic particle constitutes a core, and the polymeric covering layer constitutes a shell. The polymer which covers each surface of the magnetic particles and aggregates the particles is suitably selected depending on the purpose. Suitably used are polyacrylonitrile, a polymethyl methacrylate, a polystyrene or a copolymer thereof, which are easy to cover the magnetic particle and have acid and alkali resistances, a phenolic resin, which is superior in disperseability in water, and trialkoxysilane condensate, which strongly adheres to the magnetic particle and highly stable in water. In one or more embodiments, the average thickness of the polymeric covering layer is 0.01 to 0.25 μm. The thickness of the polymeric covering layer can be measured by optical microscopic or scanning electron microscopic (SEM) observation. Preferably, however, the average thickness of the polymeric covering layer can be accurately obtained by heating the magnetic particles covered with a polymer at a high temperature to thermally decompose the polymer to obtain the weight loss, i.e., the weight of the polymeric covering layer, and calculating the average thickness of the polymeric covering layers from the obtained weight of the polymeric covering layer and the specific surface areas of the magnetic particles.

The polymer-covering magnetic particles can be prepared by, for example, dissolving the polymer in an organic solvent which can dissolve the polymer to provide a polymer solution, dispersing magnetic particles in the polymer solution to provide a magnetic particle-dispersed polymer solution, and spraying the magnetic particle-dispersed polymer solution. In this method, the average particle size of the aggregate of the primary particles can be adjusted by adjusting the temperature at which the spray drying is carried out or spraying speed. The thickness of the polymeric covering layer in the polymer-covered magnetic particle can be determined from the mixing ratio of the polymer and the magnetic particles, the density of the polymer and the specific surface area of the magnetic particle. That is, the volume of the polymer used can be calculated from the weight and density of the polymer. The calculated volume of the polymer is divided by the surface areas of the magnetic particles calculated from the weight and the specific areas of the magnetic particles to previously determine the average thickness of the polymer. The controlling of the particle size of the polymer-covered magnetic particles varies depending on the kind of the spraying liquid (polymer solution) and a spraying method. However, to make the aggregates small in size, the size of the droplets to be sprayed and dried is made small. For example, when the spraying pressure of the spray nozzle is higher, when the spraying speed is lower, or when the rotation speed of the spray disk is higher, the particle size of the aggregates produced becomes smaller.

The size of the particulate filter aid is not particularly limited as long as the filter aid can be dispersed in water. However, a preferable range of the size exists in view of the seepage flow rate and disperseability in water. Since water flows through the spaces between the particles of the particulate filter aid, a sufficient seepage flow rate can be obtained when the particulate filter aid has a particle size of 1 μm or more. In addition, when disperseability in water is taken into consideration, particles having a particle size of 5 μm or less are low in sedimentation speed and are uniformly dispersed in water, which are preferable. As can be seen from above, the particulate filter aid preferably has a particle size of 1 to 5 μm in one embodiment.

The average particle size of the particulate filter aid can be measured by a laser diffraction method. Specifically, it can be measured by a particle size analyzer SALD-DS 21 (trade name) manufactured by Shimadzu Corporation.

In one or more embodiments, the particulate filter aid is used in an amount one to 20 times the concentration of microorganisms in the aqueous suspension of microorganisms to be concentrated.

In one or more embodiments, the particulate filter aid initially charged in the filter aid accommodating tank 14 may be in the form of powder, of a paste containing a small amount of water or of an aqueous slurry. As described above, since the particulate filter aid separated from the microorganisms in the magnetic separation device is returned to the filter aid accommodating tank 14 in the form of an aqueous slurry, the particulate filter aid initially charged in the filter aid accommodating tank 14 is suitably in the form of the paste or aqueous slurry.

<Mixing Device>

The mixing device 11 uniformly mixes the aqueous suspension of microorganisms with the particulate filter aid supplied from the filter aid accommodating tank 14. The shape, volume and material of the mixing tank 111 are not particularly limited. In the mixing tank 111, a baffle plate may be disposed in order to perform more uniform mixing. Also, in the mixing tank 111, a level sensor may be arranged if necessary. The mixing time of the aqueous suspension of microorganisms and the filter aide is sufficient for them to be mixed uniformly, and one minute or more may suffice for mixing them.

<Filtering Device>

The filtering device 12 provides the filter cake of the particulate and the microorganisms. Accordingly, the filtering device 12 is usually provided with the filter medium 121 which supports the filter cake FC. The filtration may be carried out under reduced pressure or under pressure. In the filtration under reduced pressure, the line L2 is connected to a vacuum device (not illustrated). In the filtration under pressure, the pressurization may be effected by means of the pump P1. In view of the filtering treatment speed, the filtration under pressure is preferable. In the filtration under pressure, the aqueous slurry of the microorganisms and particulate filter aid prepared in the mixing tank 111 may be supplied to the filtering device 12 under a pressure of, for example, 0.001 MPa to 1 MPa by means of the pump P1. Incidentally, a filtration which does not form a filter cake, such as an upward flow filtration, can not be used. In one or more embodiments, a high speed filtering treatment is carried out by using a particulate filter aid slightly larger than or smaller than microorganisms to form a layer of the particulate filter aid around the microorganism to suppress the deformation of the microorganisms, and pass the water through between the filter aid particles.

Without the particulate filter aid, the high speed filtering treatment can not be conducted since the microorganisms, which are readily deformed, agglomerate with each other and clog the water passageways. Further, in the case of a filtering device in which a filter medium (filter plane) is vertically arranged, and water is flowed in a direction perpendicular to the filter medium, the filter cake may often be broken up, resulting in that control of the filtration may become difficult. Therefore, a filtering device is preferably used, in which a filter medium is arranged horizontally, and water is passed downwardly through the filter medium in a direction perpendicular to the filter medium.

The filter medium 122 has sufficiently small apertures to deposit thereon the filter cake FC constituted by the particulate filter aid and the microorganisms.

<Magnetic Separation Device>

The filter cake which has been washed out from the filtering device 12 and supplied to the separation tank 131, in the form of aqueous slurry is separated into the particulate filter aid and the microorganisms by the action of the electromagnet 132 as described above. In this case, a pH-adjusting agent or a salt may be added in order to effectively separate the microorganisms and the particulate filter aid.

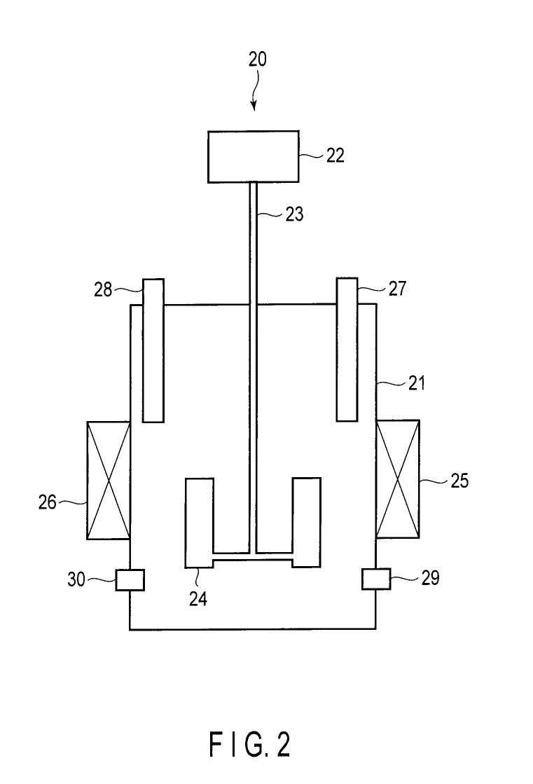

FIG. 2 illustrates another example of a magnetic separation device which may be used instead of the magnetic separation device 13 in the microorganism concentrating apparatus 10.

The magnetic separation device 20 illustrated in FIG. 2 comprises a hollow cylindrical separation tank 21 having a bottom. In the separation tank 21, a stirring mechanism is arranged. The stirring mechanism comprises a rotationally driving device 22, a stirring shaft 23 supported by the driving device 22, and stirring blades 24 provided at the tip end of the stirring shaft 23. On the outer peripheral wall of the separation tank 21, electromagnets 25 and 26 are arranged, which are connected to a power source not illustrated. Further, water inlets 27 and 28 are provided on the upper portion of the separation tank 21. The wash-out liquid containing the filter cake discharged from the filtering device 12 (FIG. 1) is supplied through one water inlet 27, and a small amount of water (containing microorganisms) for washing the particulate filter aid is supplied from the culturing tank 15 (FIG. 1) through the other water inlet 28 (that is, the water inlet 27 is connected to the line L3 of the apparatus of FIG. 1, while the water inlet 28 is connected to the line L9 of the apparatus of FIG. 1). Further, water outlets 29 and 30 are provided at lower portions of the side wall of the separation tank 21, respectively. The aqueous slurry of the concentrated microorganisms is discharged from one water outlet 29, while the particulate filter aid slurried with a small amount of the washing water from the water inlet 28 is discharged from the other water outlet 30 (that is, the water outlet 29 is connected to the line L4 of the apparatus illustrated in FIG. 1, while the water outlet 30 is connected to the line L5 of the apparatus illustrated in FIG. 1).

Incidentally, in the apparatuses illustrated in FIGS. 1 and 2, a permanent magnet can be used instead of the electromagnet. In this case, the permanent magnet can be fixed on a piston cylinder provided outside the separation tank, and the separation and recovery are effected by changing the distance between the permanent magnet and the outer wall of the separation tank. More specifically, by approaching the permanent magnet to the separation tank by means of the piston cylinder, the particulate filter aid can be held and fixed, while by pulling the permanent magnet away from the separation tank, the particulate filter aid can be released from the magnetic field.

According to the microorganism concentrating apparatus and method according to one or more embodiments described above, expeditious concentration of microorganisms can be carried out by filtering microorganisms together with particulate filter aid to make a filter cake, which is magnetically separated into the microorganisms and the particulate filter aid.

### Examples

A microorganism concentrating tests were conducted using the concentration apparatus illustrated in FIG. 1.

E. coli having an average particle size of 2 μm, and chlorella having an average particle size of 7 μm were used as the microorganisms. Magnetite particles having an average particle size of 0.2 μm, 1 μm and 2 μm, as well as Mn—Mg—Sr ferrite particles having an average particle size of 4 μm, 8 μm and 30 μm were used as the particulate filter aid and were placed in the filter aid accommodating tank 14 as an aqueous slurry having a predetermined concentration. On the other hand, an aqueous suspension of the microorganisms adjusted to a microorganism concentration of 1000 mg/L (wet basis) was place in the culturing tank 15. A filter cloth having apertures of about 3 μm was used as the filter medium 122.

Then, the suspension of the microorganisms was supplied into the mixing tank 111, while the particulate filter aid was supplied from the filter aid accommodating tank 14 into the mixing tank 111 such that the concentration of the filter aid became 10000 mg/L. The flow rate was adjusted so that he filtration rate became 2 m/h or more in linear velocity and the filtration was conducted until a filter cake 20 mm in thickness was deposited on the filter medium 121. After the filtration, the filter cake was washed out from the filter medium with water in an amount 0.1 times the amount of the filtered water. Thus, an aqueous slurry having a microorganism concentration increased by 10 times was introduced into the separation tank 131. While the slurry was stirred with the stirrer 133, the electromagnet 132 was activated to attract and fix the particulate filter aid, and the concentrated slurry of the microorganisms was recovered. Further, the same amount of water was supplied to slurry the particulate filter aid from which the microorganisms had been separated was supplied to the filter aid accommodating tank 14. It was confirmed that the recovered magnetic particles could be re-used without problem.

The test results are shown in Table 1 below in terms seepage flow rate in linear velocity. Incidentally, similar test was conducted without using particulate filter aid, for comparison.

TABLE 1

| | |
|---|---|
| Magnetic particles | Seepage flow rate |

| | Average | E. coli | Chlorella |
|---|---|---|---|
| | particle | (Average | (Average |
| Kind | size | size: 2 μm) | size: 7 μm) |
| None | — | X | X |
| Magnetite | 0.2 μm | Δ (*) | Δ (*) |
| Magnetite | 1 μm | ◯ | ◯ |
| Magnetite | 2 μm | ◯ | ◯ |
| Mn—Mg—Sr | 4 μm | ◯ | ◯ |
| ferrite | | | |
| Mn—Mg—Sr | 8 μm | Δ | ◯ |
| ferrite | | | |
| Mn—Mg—Sr | 30 μm | X | Δ |
| ferrite | | | |

In Table 1, the symbols have the following meanings:
(*): Some magnetic particles passed through the filter medium
◯: Linear velocity of 2 m/h or more
Δ: Linear velocity of 0.5 m/h or more, but less than 2 m/h
X: Linear velocity of less than 0.5 m/h, or filtration impossible

As is apparent from Table 1, when the particulate filter aid (magnetic particles) was not used, the microorganisms clogged the apertures of the filter cloth, and the filtration was impossible. A linear velocity of 2 m/h or more could be achieved for both of E. coli and chlorella, when the particulate filter aid (magnetic particles) having an average particle size of 1 μm or more was used (note, however, that some magnetic particles passed through the filter cloth for both of E. coli and chlorella, when magnetic particles having an average particle size of 0.2 μm were used). Further, when a filter aid having a particle size of up to 4 μm in the case of E. coli, and when a filter aid having a particle size of up to 8 μm in the case of chlorella, a linear velocity of 2 m/h or more could be achieved. However, when a further larger filter aid was used, the linear velocity was lowered. Thus, it has been revealed that when a filter aid having an appropriate particle size is added for the filtration, concentrated microorganisms can be expeditiously obtained.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A microorganism concentrating apparatus comprising:
a mixing device configured to mix an aqueous suspension of microorganisms with a particulate filter aid comprising magnetic particles to prepare an aqueous slurry of the microorganisms and the particulate filter aid;
a filtering device configured to filter the aqueous slurry supplied from the mixing device to provide a filter cake comprising the microorganisms and the particulate filter aid, and a filtrate; and
a magnetic separation device configured to magnetically separate the filter cake supplied from the filtering device into the microorganisms and the particulate filter aid.

2. The apparatus according to claim 1, wherein the particulate filter aid has an average particle size 0.1 to 10 times that of the microorganisms.

3. The apparatus according to claim 2, wherein the particulate filter aid consists of the magnetic particles.

4. The apparatus according to claim 2, wherein the particulate filter aid consists of aggregates of magnetic particles whose surfaces are covered with a polymer.

5. The apparatus according to claim 1, wherein the filtering device comprises an envelope and a horizontally arranged filter medium partitioning the inner space of the envelope into an upper space into which the aqueous slurry is supplied and a lower space from which the filtrate is discharged, wherein the filter cake is formed on the filter medium.

6. The apparatus according to claim 1, wherein the magnetic separation device comprises a tank which receives the filter cake supplied from the filtering device, and an electromagnet arranged on the outer wall of the tank.

7. The apparatus according to claim 5, further comprising an aqueous wash-out liquid supply line configured to supply an aqueous wash-out liquid to the filter cake, thereby washing out the filter cake from the filter medium.

8. The apparatus according to claim 7, further comprising a filter cake supply line supplying the washed-out filter cake together with the aqueous wash-out liquid to the magnetic separation device.

9. The apparatus according to claim 1, wherein the magnetic particles are formed of magnetite.

10. The apparatus according to claim 1, wherein the microorganisms comprises E. coli, waterbloom or chlorella.

11. A microorganism concentrating method comprising:
mixing an aqueous suspension of microorganisms with a particulate filter aid comprising magnetic particles to prepare an aqueous slurry of the microorganisms and the particulate filter aid;
filtering the aqueous slurry through a filter medium to provide a filter cake comprising the microorganisms and the particulate filter aid, and a filtrate, wherein the filter cake is provided on the filter medium; and
magnetically separating the filter cake into the microorganisms and the particulate filter aid.

12. The method according to claim 11, wherein an aqueous wash-out liquid is flowed toward the filter cake to wash out the filter cake from the filter medium, thereby preparing an aqueous slurry comprising the washed-out filter cake, which is subjected to the magnetic separation.
